(19) United States
(12) Patent Application Publication
(10) Pub. No.: US 2006/0078513 A1
(43) Pub. Date: Apr. 13, 2006

(54) Oral care system

(76) Inventor: William T. Wilkinson, Salem, NJ (US)

Correspondence Address: Charles S. Knothe, Esquire, 3516-14 Silverside Road, Wilmington, DE, 19810, US

(21) Appl. No.: 10/962,526

(22) Filed: Oct. 13, 2004

(51) Int. Cl. A61K 8/22

(52) U.S. Cl. 424/53

(57) ABSTRACT

This invention discloses a multi-purpose oral hygiene system for the mouth and teeth with a liquid tooth-whitening product composed of natural ingredients that can be swallowed mixed with a liquid breath freshener in a container to hold and deliver the tooth-whitening product and breath freshener to the mouth and teeth. Flavors, colors and anti-bacterials can be added to the combination of liquid tooth-whitening and liquid breath freshener. The delivery means can be a portable spray container. The spray container can be a non-pressurized or a pressurized dispenser.

### BACKGROUND OF INVENTION

The current market place includes many different types of liquids or oral care including: antiseptics, antibacterial, breath fresheners, mouth washes and mouth rinses. Most recently, there has been considerable emphasis on whitening product for the teeth. Most of these products are applied as a gel or paste. An applicator is used such as a brush, mold, or tray. The whitener is usually applied for a considerable time period and the process is involved and time consuming. Usually it is done at home or in a dentist's office.

There is a need for a way that a tooth whitener can be applied quickly and easily, often, and has desirable properties. The present invention is a quick and easy way to whiten teeth that can be done conveniently anywhere. Preferably, it is a liquid that can be applied by spray or squirting on the teeth. It can also be a paste that can be squeezed into the mouth and applied with the tongue. Preferably the whitener is composed of natural ingredients, that are not harmful and can be swallowed or ingested.

The whitener ingredient is combined with breath freshener. The freshener can be of different flavors, tastes and colors. Further, this combination can be enhanced with antiseptics, anti-bacterials, substances to sooth sore throats, and sweeteners so that it has a candy flavor to enhance the appeal. The combination of ingredients freshens the breath, kills germs, cleans the tongue and can be a topical sore throat treatment and can assist in the healing of mouth sores. The invention can use only the tooth-whitening product.

The whitener and breath fresheners are preferably delivered by a small, portable spray container, or alternatively a flexible squirt bottle or micro-atomizer. The use of this delivery method is advantageous because the whitening can be done anywhere. With the spray container it is easy and quick to apply, and compact and portable to carry or store. And, finally it is simple to apply. The top of the spray container can be of differing colors to represent differing flavors or ingredients.

### BRIEF SUMMARY OF THE INVENTION

The invention is a multi-purpose oral hygiene system for the mouth and teeth which have a liquid tooth-whitening product composed of natural ingredients that can be swallowed mixed with a liquid breath freshener or mouthwash in a container to hold and deliver the tooth-whitening product and breath freshener to the mouth and teeth. An anti-bacterial can be added to the combination of liquid tooth-whitening and liquid breath freshener. Flavors and colors can be added to the combination of liquid tooth-whitening and liquid breath freshener. Further, ingredients to soothe sore throats are added to the combination of liquid tooth-whitening and liquid breath freshener.

The delivery means can be a portable spray container. The spray container can be a non-pressurized pump dispenser or a pressurized pump dispenser or combination of both such as a squeeze bottle. The dispenser can have an extension to reach the back of the mouth.

Rather than being a liquid the tooth-whitener and breath-freshener can be a paste.

### BRIEF DESCRIPTION OF THE DRAWINGS

FIG. 1 is perspective view of the squeeze dispenser without the cap.

FIG. 2 is the front view of the squeeze dispenser.

FIG. 3 is the front view of the squeeze dispenser with the cap.

FIG. 4 is a perspective view of the pump dispener with the cap.

FIG. 5 is a perspective view of the pump dispeners with the cap separated from the container.

FIG. 6 is a front view of the pressurized dispenser without the cap.

FIG. 7 is the front view of the pressurized dispenser with the cap.

### DETAILED DESCRIPTION OF THE PREFERRED EMBODIMENT

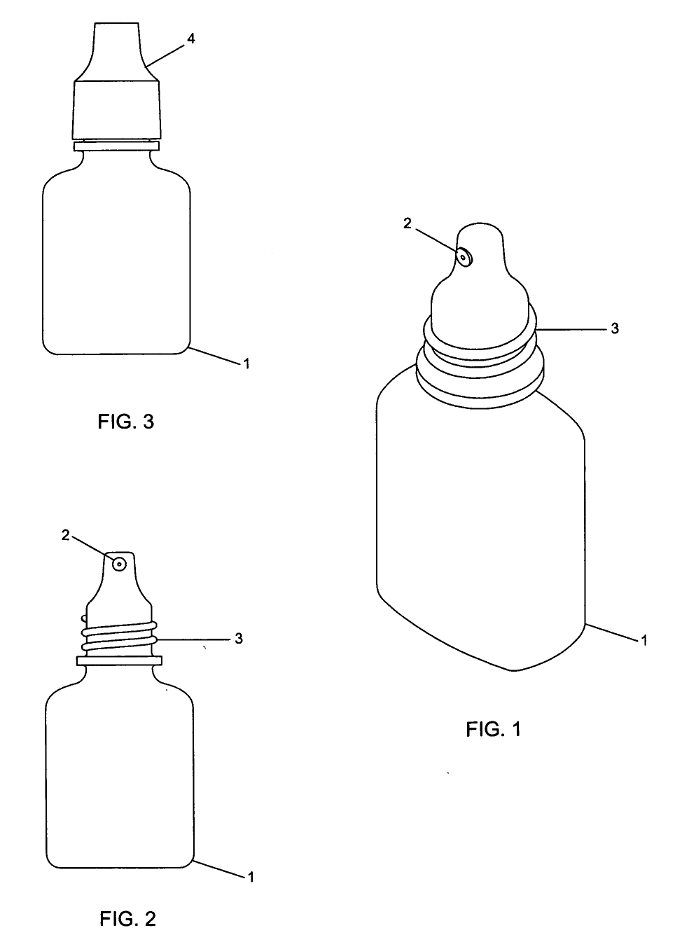

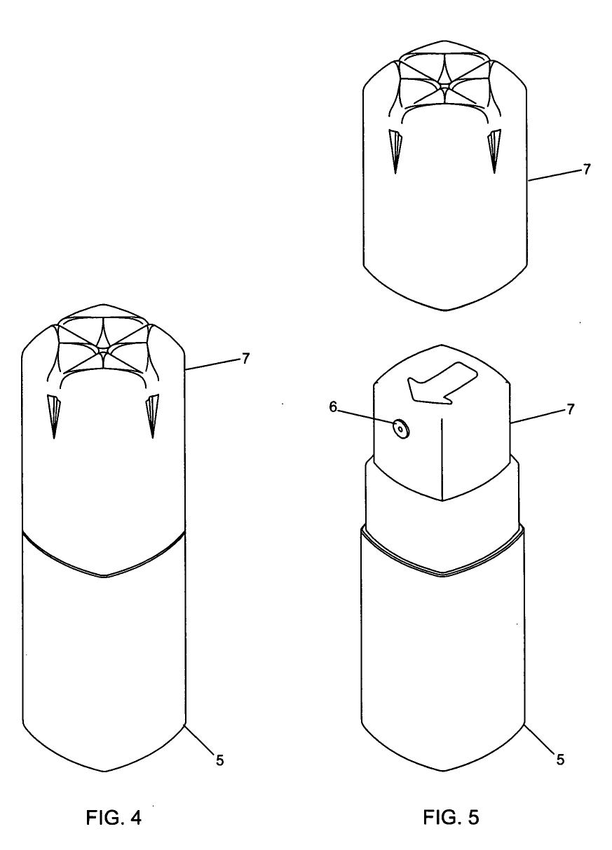

FIG. 1 shows an isometric view of the squeeze spray dispenser used to deliver the tooth-whitener and breath-freshener. 1 is a squeezable bulb that contains the tooth-whitener and breath-freshener. The nozzle 2 is located at the top of the dispenser and directs the stream of tooth-whitener and breath-freshener at the user's teeth and into the user's mouth. The screw thread 3 are used to mate with the treads in the cap. FIG. 2. is a front view of the squeezable bulb 1 with the nozzle 2. FIG. 3 depicts the squeeze bulb 1 with the cap 4 threaded on to it.

The tooth-whitener can contain but is not limited to hydrogen peroxide, potassium hydroxide, phosphoric acid, sodium bicarbonate, sodium pyrophosphate, tetra sodium pyrophosphate, resinous glaze, carnauba wax, or sodium hydroxide.

FIG. 4 shows the manually operated pressure sprayer for dispensing the tooth-whitener and breath-freshener. The container 5 holds the tooth-whitener and breath-freshener and the cap 7 is attached. FIG. 5 shows the plunger 7 that when depressed forces the tooth-whitener and breath-freshener through the nozzle 6. In FIG. 5 the cap 7 is removed from the base 5.

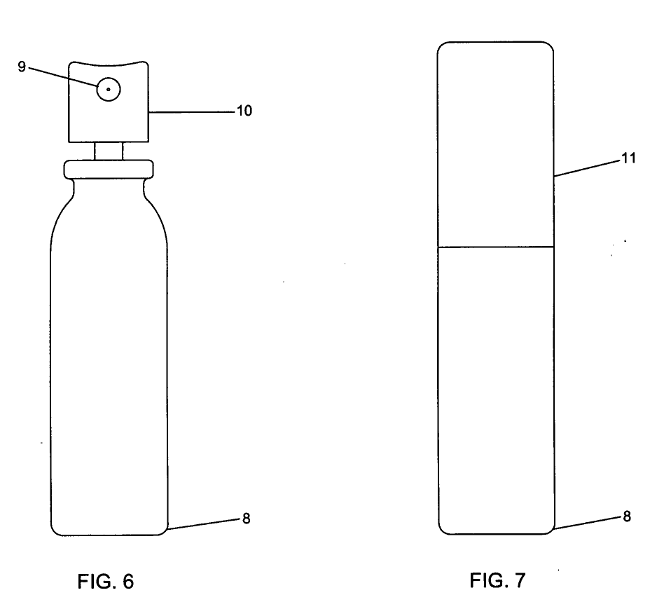

FIG. 6 is a pressured sprayer for dispensing the tooth-whitener and breath-freshener. The container 8 is the pressurized cavity that holds the tooth-whitener and breath-freshener and the propellant. When the plunger 10 is depressed the propellant forces the tooth-whitener and breath-freshener through the nozzle 9. FIG. 7 shows the cap 11 attached to the container 8.

I claim a single or multi-purpose oral hygiene system for the mouth and teeth comprising:

1. A liquid tooth-whitening product composed of natural ingredients that can be swallowed,
A liquid breath freshener and/or mouthwash mixed with the tooth-whitening product and
A means to hold and deliver the tooth-whitening product and breath-freshener to the mouth and teeth.

2. The same invention as in claim 1 wherein only a tooth-whitening product is used.

3. The same invention as in claim 1 wherein liquid antiseptics or anti-bacterial is added to the combination of liquid tooth-whitening and liquid breath-freshener.

4. The same invention as in claim 1 wherein flavors are added to the combination of liquid tooth-whitening and liquid breath freshener.

5. The same invention as in claim 1 wherein the combination of liquid tooth-whitening and liquid breath-freshener is colored different colors.

6. The same invention as in claim 1 wherein the ingredients to soothe sore throats are added to the combination of liquid tooth-whitening and liquid breath-freshener.

7. The same invention as in claim 1 wherein the delivery means is a portable spray container.

8. The same invention as in claim 1 wherein the delivery means is a non-pressurized pump dispenser.

9. The same invention as in claim 1 wherein the delivery means is a pressurized dispenser.

10. The same invention as in claim 1 wherein the delivery means is a squeeze bottle.

11. The same invention as in claim 1 wherein the delivery means has a extension to reach the back of the mouth.

12. The same invention as in claim 1 wherein the tooth-whitener is a paste.

13. The same invention as in claim 1 wherein the breath-fresher is a paste.

14. The same invention as in claim 1 wherein the tooth-whitening product can contain but is not limited to hydrogen peroxide, potassium hydroxide, phosphoric acid, sodium bicarbonate, sodium pyrophosphate, tetra sodium pyrophosphate, resinous glaze, carnauba wax, or sodium hydroxide.

15. The same invention as in claim 1 wherein the breath-freshener can contain common ingredients but not limited to alcohol, poloxamer 407, methylparaben, sodium saccharin, propyparaben, glycerin, polysorbate 80, sodium saccharin, sodium benzoate, cetylpyridinium chloride, domiphen bromide, benzoic acid, thymol, eucalyptol, methyl salicytate, methol and sorbital solution.
